(19) United States
(12) Patent Application Publication
(10) Pub. No.: US 2021/0315516 A1
(43) Pub. Date: Oct. 14, 2021

(54) Novel Approach using for Generating Diagnoses using Natural Language Processing Algorithms for Speech Disorders and a Modern Use of Voice Assistant Systems for Stuttering and Rhotacism Therapy

(71) Applicants: Neeraj Rattehalli, East Palo Alto, CA (US); Ishan Jain, Fremont, CA (US)

(72) Inventors: Neeraj Rattehalli, East Palo Alto, CA (US); Ishan Jain, Fremont, CA (US)

(21) Appl. No.: 16/842,758

(22) Filed: Apr. 8, 2020

(51) Int. Cl. A61B 5/00; G10L 15/07; G10L 25/66; G16H 50/20

(52) U.S. Cl. CPC A61B 5/4803; G16H 50/20; G10L 25/66; G10L 15/07

(57) ABSTRACT

A method that includes novel algorithms to biomedical analysis, specifically including the treatment of stuttering, rhotacism, and stutter pauses. Additionally, it provides systematic methods to collect data and treat the patients given the initial data. All the analysis and data collection are done through the convenience of a voice assistant, like Amazon Alexa or Google Home, or through a phone or web application. The claims presented in this disclosure secures the entire combined system and its individual components.

### FIELD OF THE DISCLOSURE

The present disclosure generally relates to biomedical analysis with machine learning. More particularly, the present disclosure relates to systems and methods for speech disorder treatment such as stuttering and rhotacism therapy through natural language processing.

### BACKGROUND OF THE DISCLOSURE

Stuttering is a speech disorder associated with interruptions in the flow of speech due to unintentional repetitions and elongations of sounds, syllables, words, or phrases or involuntary pauses in a speech. Over seventy million people stutter globally. In order to mitigate and treat one's stutter, several meet with stutter therapists who have been trained to assess and offer support to patients. However, traditional treatment is costly, and at times, hard to find. Since therapists work under a time constraint, they are often limited by monetary intentions and personal biases towards their diagnosis. Additionally, most providers fail to cover stuttering therapy under present insurance policies.

The social and psychological impacts of stuttering can detriment one's life in multiple ways. For students in elementary, middle, and high school, stuttering can entail bullying and mistreatment, leading to mental health disorders. Since there is presently no cure for a stutterer, one must either learn to live with their stutter or face the high fees of therapists. For over 25% of stutterers, they will continue to stutter into adulthood. In adults, stuttering may come off as unprofessional and can take away from a speaker's credibility. Stuttering can cause insecurity due to the prejudices associated with speech slurs.

In the current market, there are few to none applications that treat, diagnose, and provide custom exercises in response to stutters. Present applications simply record user speech sessions and share it with a therapist. However, this still requires a therapist, which is the most expensive part of the treatment. The only real benefits of such apps are streamlining the process and communication with stutter therapists. In order to progress in developments with stuttering therapy and reduce the cost of therapy, one must look to machine learning algorithms to assess speech and generate relevant feedback about mispronounced and misspoken word segments, as well as inaudible gaps of speech.

Thus, there is a need for an artificially intelligent approach for generating diagnoses and treatments for stuttering patients.

### BRIEF SUMMARY OF THE DISCLOSURE

This section will be the CLAIMS at the end rewritten in paragraph form once finalized

This patent makes claims towards a self-contained system that can assess, diagnose, and treat stutters and rhotacism in patients through a voice assistant, a web-based assistant, or an application on the phone. Essentially, the patent secures ownership over the algorithmic use of computing technology to treat stutters. Additionally, the patent includes the proprietary and novel algorithms for diagnosing the stutters, determining stutter pauses, assessing rhotacism, and matching patients with speech exercises given the diagnosis.

For the stutter diagnosis algorithm, the present disclosure claims the hyphenation followed by a differing algorithm which compares consecutive syllables. If there are too many repeated syllables. The algorithm will be able to pinpoint the repeated syllable.

For the stutter pauses algorithm, this patent claims the methodology to split vocal input data into periods of silence and sounds using a decibel threshold. While doing so, the algorithm can identify prolonged periods of silence during speech, compare it to normal speech pauses, and then determine the neighboring syllables that caused the auditory pause using the aforementioned hyphenation algorithm.

For the rhotacism diagnosis algorithm, the present disclosure claims the hyphenation followed by a differing algorithm which compares the spoken syllables to the true syllables. After overlaying the common syllables and extracting the incorrect ones, the algorithm has successfully determined mispronounced syllables.

For the matching speech disorder algorithm, we will use a dictionary, with syllabized words, and randomly choose words that contain similar syllable to the words that had been pronounced incorrectly either due to a stutter or a rhotacism. As for stutter pauses, we will create random sentences using this dictionary that contain neighboring words that are likely to create pauses given the patient's initial diagnosis.

### BRIEF DESCRIPTION OF THE DRAWINGS

The present disclosure is illustrated and described herein with reference to various drawings, in which like reference numbers are used to denote like system components/method steps, as appropriate, and in which:

FIG. 1 is a visual representation of the sample training exercises provided to the patient upon input data being received, as shown by the input voice assistant at 100. Upon receiving the input data, it is preprocessed and analyzed by the algorithms at 102. Finally, using the Markov-Chain algorithm at 104, the input data is piped into the algorithm and recycled back to the frontend client. This process is effective for providing the patient with exercises to improve their speech.

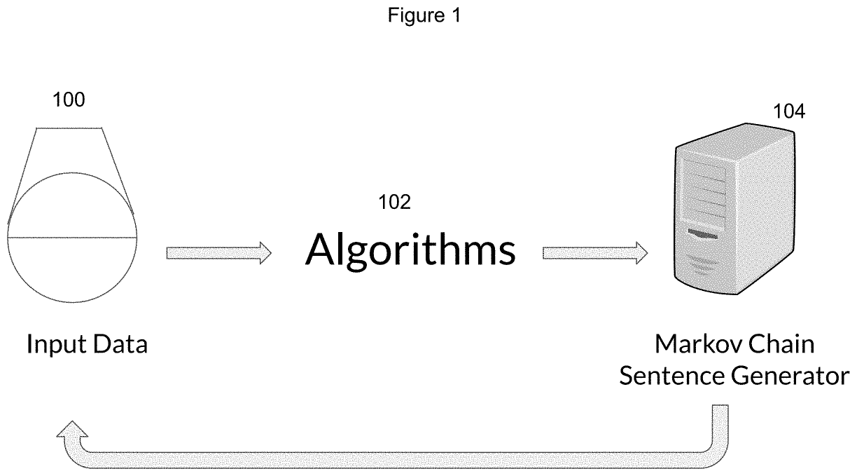

FIG. 2 is a flowchart of our stutter detection algorithm. The sound is first collected as an input at 200. Essentially, the software hyphenates sounds at 202, then it scans the hyphenated representation for repetition at 204. Then using the Markov Chain Sentence Generator at 206, exercises with the same stuttered syllables are curated. This will then be provided to the user as demonstrated by 208.

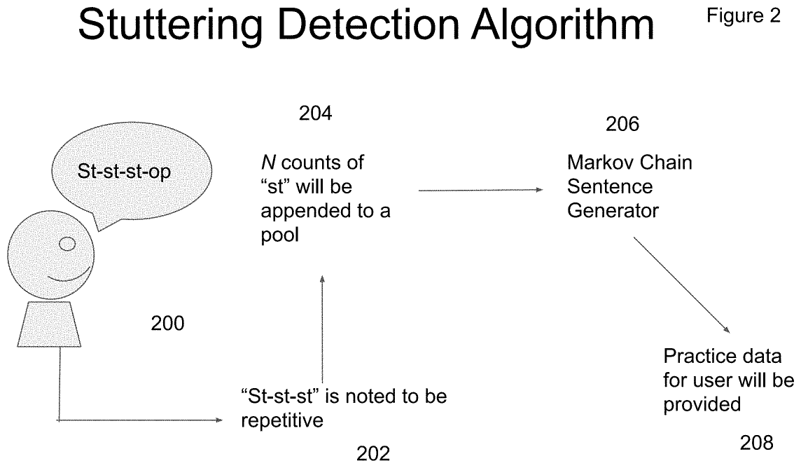

FIG. 3 is a flowchart of our rhotacism detection algorithm. The sound is first collected as an input at 300. Essentially, it autocorrects the word at 302 and then hyphenates sounds at 304. Then it hyphenates the autocorrected word at 304 as well. The algorithm proceeds to pair the hyphenated syllables at 306. When it notices a difference like at 308, it makes note of the type of rhotacism as shown by 310.

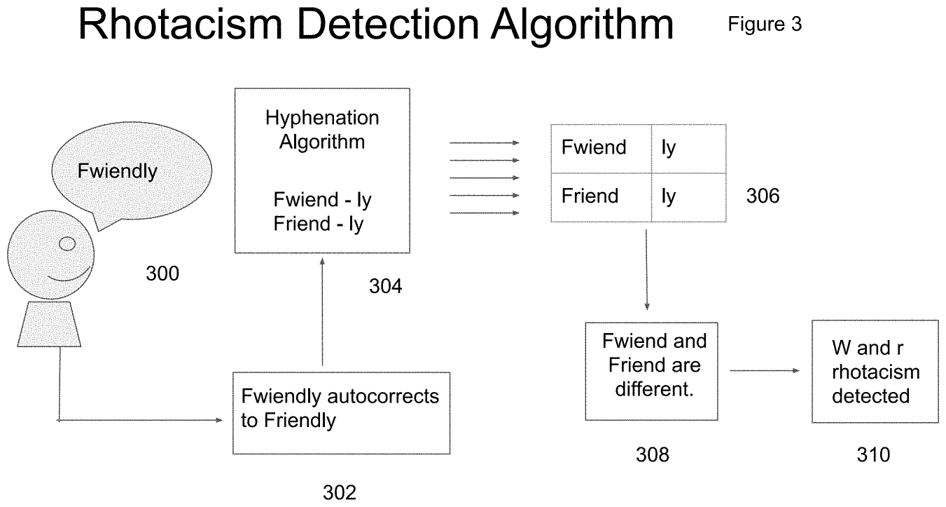

FIG. 4 is a flowchart exhibiting the mathematical modelling behind classifying and identifying stuttering disorder specifically. By gathering input data from a patient (demonstrated at 400), the program initially segments the sentences into individual words (402 representation). Between each word, the program also monitors the amount of time that the patient takes to dictate each word, shown by 404. By using inequalities, we would be available to further verify whether a patient is diagnosed with stuttering disorder, and if so, the specific mispronounced words will be bucketed into the training exercises dataset, as shown in 406, 408, and 410.

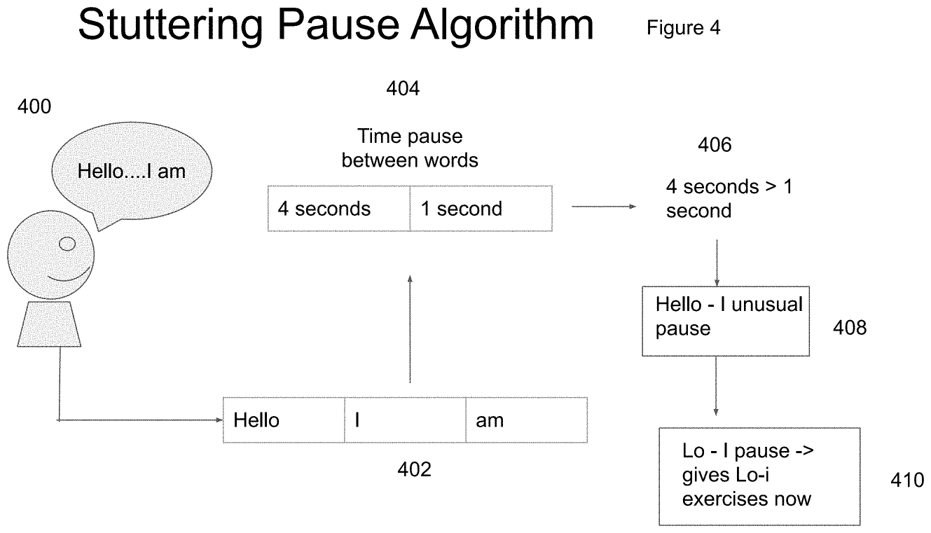

FIG. 5 represents a process that a given user undergoes throughout the diagnosis process. More specifically, the figure demonstrates the process of the Markov-Chain Sentence generator algorithm. At 500, input data is gathered through the user's vocal speech. From there, the algorithm utilizes the data to process, as shown at 502. Finally, the program outputs sample exercises that the user can begin practicing, as demonstrated by 504.

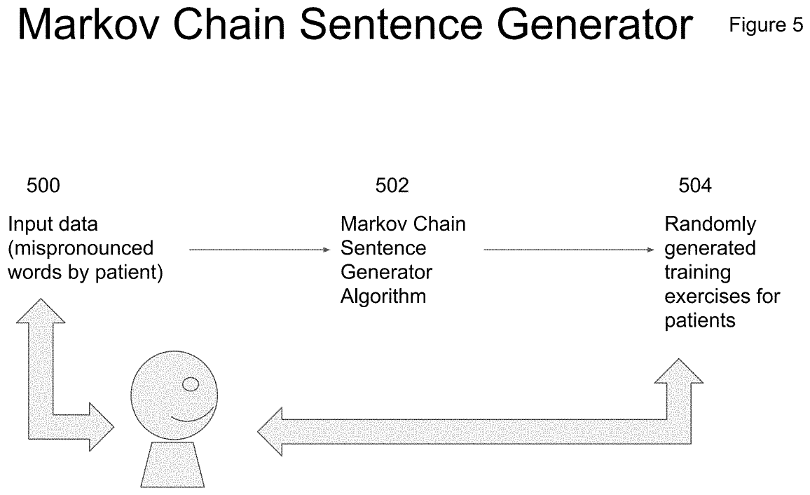

### DETAILED DESCRIPTION OF THE DISCLOSURE

In various exemplary embodiments, the present disclosure relates to systems and methods for stuttering therapy utilizing natural language processing. In particular, one inputs vocal data into a technological system that contains an audio input mechanism. A convolutional neural network is trained using crowd-sourced data from individuals with stuttering disorder from across the world. With this vocal data, a speech-to-text algorithm converts the speech into UTF-8 text format for the machine to further analyze. The natural language is executed on the newly generated text data, generating outputs. With this initial input data, the trained algorithm analyzes the speech, eventually finding errors and mispronounced words or phrases in the text. Each patient will then receive access to their vocal mispronunciations and errors, allowing them to proceed to the following step in the treatment. Utilizing web-based application program interfaces (APIs), the service queries training exercises from a public repository for the patient to practice. With a repeating loop of analysis and practice sets, near perfect accuracy and well-versed treatment is evident. The service for treating patients can provide various advantages, for example, 1) time efficiency 2) cost efficiency 3) constant improvement and specific monitoring 4) automated method for diagnosing disorder, allowing patients to self-diagnose with ease.

### Architecture of Algorithm for Rhotacism Treatment

In an exemplary embodiment, the initial process for treating rhotacism encompasses an auto-correct filter. When a patient provides input vocal data to smart home device such as a Google Home or an Amazon Echo, the speech data is converted into UTF-8 text format, which is then filtered using an algorithm for determining the intended word. Like the conventional autocorrect features in mobile and cellular devices, the autocorrect filter will determine words that closely match with the words that were originally spoken. After the speech is convoluted through, eventually, there will be two pools of data. The first pool will be the raw text data that has been converted from the speech data. The second pool encompasses the data that the autocorrect algorithm filter was able to infer and generate.

Proceeding the autocorrect filtration and data pool fabrication, another hyphenation algorithm is executed in order to achieve a separation of syllables among every single word. By iterating through the entire text with a single word at a time, this filtration algorithm will generate a more complex breakdown of every word, rendering syllables. With this breakdown, the generated text will become more simplified to further analyze. The hyphenation algorithm will also further segment each of the two pools of language into more comprehensible text. Hyphenation algorithm for breaking into syllables.

After the pre-analyses have been conducted, the service compares both pools of data. By iterating through the hyphenated words, each syllable of each pool will be compared with both iterations from the differing pools. When there is a difference between the pool with the correct words and the pronounced data, the service will render these erroneous pronounced words into a separate dictionary. Following this, the patient will be reinforced with those words to practice, allowing them to improve constantly. By reiterating over the service on a periodic basis, one could evidently improve and treat the mispronunciations and erroneous dictions that they possess.

### Architecture of Algorithm for Stuttering Treatment

In order to treat stutters, the service utilizes a syllabication or hyphenation algorithm in order to produce a single pool of vocal data in text format. A stutter is a repetition of certain syllables in a word. Thus, when a patient diagnosed with a stuttering disorder speaks, some syllables will be repeated with an n count (where n is any positive integer). In various exemplary embodiments, the algorithm utilizes this mechanism to find treatment exercises for the stuttering patients. After utilizing the speech-to-text algorithm, UTF-8 text type of data can be generated. In contrast to the rhotacism treatment algorithm, the stuttering treatment algorithm stores vocal data in text format in just one pool, instead of two. Thus, a pool with hyphenated or syllabicated words will be present in a pool of data.

With the generation of syllabicated word data, the stuttering treatment algorithm will then proceed to further analyze. By comparing a syllable with its adjacent syllables, the algorithm will be able to identify repetition in pronunciation, allowing patients to receive feedback for their statements. When two adjacent syllables are alike, then the algorithm will store the mispronounced word in a dictionary. It will consistently iterate through the entire pronounced statement(s), and the algorithm will store all words with repeated syllables in a dictionary. Thus, when the algorithm completely executes after processing, the patient will receive feedback for their pronunciation. After learning the patient's speaking style, the service generates sample training exercises for the patient, for it will generate words using a Markov chain sentence generator. With this training loop, the patient will be able to constantly improve and learn, for the service will provide sentences to practice pronouncing that encompass the words that are erroneously dictated by the patient.

With the incorporation of smart home devices such as a Google Home or an Amazon Echo, the service can further foster the ease of being accessible for stuttering patients. When a patient is resting in their home or residency, the smart home device will be present. Thus, a patient will be able to easily reach out to an artificially intelligent therapist, the smart home device. Having the service embedded in a smart home device is advantageous in multiple ways, for example, it becomes 1) easily accessible 2) cost efficient 3) more comfortable for the patient as speaking to an inhumane object is less stressful than speaking with a human therapist.

### § 3.0 Markov Sentence Generator Method

The utilization of a Markov Sentence generator allows the service to generate sample sentences for the patient to practice. When the algorithm processes and finds errors in pronunciation, the Markov Sentence Generator method will generate vocal exercises for the patient to practice with those specific words that the patient was unable to pronounce. With the incorporation of a well-versed and tested algorithm, Markov Sentence Generator, the accuracy of having sentences that can adequately provide supplemental material for patients can be achieved evidently.

### § 4.0 Random Exercise Generator Method

After diagnosis of the stuttered syllables, our novel software will generate new, unique exercises given the data. Essentially, using a database of the English words, which have been syllabized, we will create custom generated exercises. For example, stutters that are due to inaudible pauses are generally due to a pair of connected syllables. Using our English word database, we can find words with similar ending—starting syllables pair

What is claimed is:

1. A systematic method that utilizes automated tools to help assist individuals diagnosed with stutter and other rhotacism disorders with the help of one's personal voice assistant module

2. The automated systematic method of claim 1, wherein using a tool that integrates voice assistant services and devices such as a Google Home and an Amazon Echo for stuttering and rhotacism therapy wherein the patient receives access to the service with a remote device that contains smart-home-based vocal input services.

3. The automated systematic method of claim 1, wherein a method that utilizes a mobile voice assistant such as Siri, Cortona, Google Assistance, and a mobile application for stuttering and rhotacism therapy wherein the patient receives access to the service with a remote device that contains mobile capabilities and remote functionality.

4. The automated systematic method of claim 1, wherein a web-based application is optimally used with a voice integration system for active stuttering and rhotacism therapy wherein the patient receives access to the service with a remote device that contains web-based capabilities.

5. The automated systematic method of claim 1, where in a natural language and mathematical-based algorithm for determining rhotacism processes data—i.e. hyphenation and autocorrect algorithm matched with an ultimate differing algorithm for providing feedback.

6. The automated systematic method of claim 1, where in an algorithm for determining stutters with repeated syllables is deployed, harnessing the capabilities of natural language processing and linguistic analysis.

7. The automated systematic method of claim 1, wherein a mathematical-based algorithm for determining stutter pauses in between pronunciation by factoring in aspects such as vocal frequency in terms of words spoken comes into factor.

8. The automated systematic method of claim 1, wherein a algorithm for matching stutters with exercises is executed, wherein the patient receives feedback for his/her vocal performance. Harnessing the capabilities of the Markov sentence generator methodology, such an endeavor can be achieved.

9. A novel and remote approach using modern technological services to automating stuttering, rhotacism, and other speech disorders using adaptable natural language processing methods and techniques.

10. The application of claim 9, wherein the unique adaptation and inclusion of natural language processing into the study and field of bioinformatics.

11. The application of claim 9, wherein the adaptation and unique utilization of a comparison algorithm to differentiate between several pools of vocal data that is structured and arranged in randomized orders.

12. The application of claim 9, wherein the adaptation of a novel method for the inclusion of voice assistants and services for the initial collection and pooling of vocal data for biomedical analysis and further treatment.
